(19) United States
(12) Patent Application Publication
(10) Pub. No.: US 2021/0383923 A1
(43) Pub. Date: Dec. 9, 2021

(54) POPULATION-LEVEL CARE PLAN RECOMMENDER TOOL

(71) Applicant: KONINKLIJKE PHILIPS N.V., EINDHOVEN (NL)

(72) Inventors: Reza SHARIFI SEDEH, Malden, MA (US); Eran SIMHON, Cambridge, MA (US)

(21) Appl. No.: 17/283,684

(22) PCT Filed: Oct. 9, 2019
(86) PCT No.: PCT/EP2019/077299

§ 371 Date: Apr. 8, 2021

Related U.S. Application Data
(60) Provisional application No. 62/744,443, filed on Oct. 11, 2018.

(51) Int. Cl. G16H 50/20; G16H 50/30; G16H 50/70; G16H 40/20; G16H 10/60; G16H 15/00; G06N 20/00; G06F 3/0482

(52) U.S. Cl. CPC G16H 50/20; G16H 50/30; G16H 50/70; G06F 3/0482; G16H 10/60; G16H 15/00; G06N 20/00; G16H 40/20

(57) ABSTRACT

A care plan tool for defining care plans for patients with constrained care plan resources, including: a patient clustering and risk stratification module configured to cluster a group of patients into patient cohorts and configured to produce a machine learning model to predict the risk of a medical condition for each patient cohort based upon medical data for the patients in the cohort; an optimization module configured to determine an optimized care plan for each patient cohort based upon the machine learning models for each patient cohort and the constrained care plan resources; a matching care plan module configured to receive patient data for new patients and configured to match the new patients to a patient cohort and associated care plan; and a new care plan optimization module configured to receive patient specific constraints for new patients from a care manager and configured to determine a new optimized care plan for each new patient based upon the machine learning models for each patient cohort, the constrained care plan resources, patient data for new patients, and patient specific constraints.

### TECHNICAL FIELD

Various exemplary embodiments disclosed herein relate generally to a population-level care plan recommender tool.

### BACKGROUND

Descriptive, diagnostic, and predictive analytics have been used significantly to understand the healthcare system. For example, the risk of re-admission, emergency department (ED) visits, mortality, and annual healthcare expenditure has been widely studied. These tools have not been used to determine how a department such as the ED may use its resources to best benefit its patients.

### SUMMARY

A summary of various exemplary embodiments is presented below. Some simplifications and omissions may be made in the following summary, which is intended to highlight and introduce some aspects of the various exemplary embodiments, but not to limit the scope of the invention. Detailed descriptions of an exemplary embodiment adequate to allow those of ordinary skill in the art to make and use the inventive concepts will follow in later sections.

Various embodiments relate to a care plan tool for defining care plans for patients with constrained care plan resources, including: a patient clustering and risk stratification module configured to cluster a group of patients into patient cohorts based upon cohort criteria and configured to produce a machine learning model to predict the risk of a medical condition for each patient cohort based upon medical data for the patients in the cohort; an optimization module configured to determine an optimized care plan for each patient cohort based upon the machine learning models for each patient cohort and the constrained care plan resources; a matching care plan module configured to receive patient data for new patients and configured to match the new patients to a patient cohort and associated care plan; and a new care plan optimization module configured to receive patient specific constraints for new patients from a care manager and configured to determine a new optimized care plan for each new patient based upon the machine learning models for each patient cohort, the constrained care plan resources, patient data for new patients, and patient specific constraints.

Various embodiments are described, further including a first input GUI configured to receive from a care manager one of rules, constraints, and preferences to define the constrained care plan resources.

Various embodiments are described, further including a data digestion module configured to obtain patient data from a patient database.

Various embodiments are described, wherein the patient database includes one of electronic medical records, zip code data, care provider data, and constraints data.

Various embodiments are described, further including a database of optimized care plan for each patient cohort.

Various embodiments are described, further including a patient data digestion model configured to extract patient data for new patients from a patient data base.

Various embodiments are described, further including a second input GUI configured to present the optimized care plan for each new patient to a care manager.

Various embodiments are described, wherein the second input GUI is configured to receive patient specific constraints for new patients from a care manager.

Various embodiments are described, wherein the optimization module uses mixed-integer optimization to determine the optimized care plan for each patient cohort.

Various embodiments are described, wherein the new care plan optimization module uses mixed-integer optimization to determine the optimized care plan for each patient cohort.

Further various embodiments relate to method for defining care plans for patients with constrained care plan resources, including: clustering, by a patient clustering and risk stratification module, a group of patients into patient cohorts based upon cohort criteria; producing, by the patient clustering and risk stratification module a machine learning model to predict the risk of a medical condition for each patient cohort based upon medical data for the patients in the cohort; determining, by an optimization module, an optimized care plan for each patient cohort based upon the machine learning models for each patient cohort and the constrained care plan resources; receiving, by a matching care plan module, patient data for new patients; matching, by a matching care plan module, the new patients to a patient cohort and associated care plan; receiving, by a new care plan optimization module, patient specific constraints for new patients from a care manager; and determining, by a new care plan optimization module, a new optimized care plan for each new patient based upon the machine learning models for each patient cohort, the constrained care plan resources, patient data for new patients, and patient specific constraints.

Various embodiments are described, further including receiving, by The method of claim 11, from a care manager one of rules, constraints, and preferences to define the constrained care plan resources.

Various embodiments are described, further including obtaining, by a data digestion module, patient data from a patient database.

Various embodiments are described, further including the patient database includes one of electronic medical records, zip code data, care provider data, and constraints data.

Various embodiments are described, further including the optimized care plan for each patient cohort is stored in a database of optimized care plans.

Various embodiments are described, further including extracting, by a patient data digestion model, patient data for new patients from a patient data base.

Various embodiments are described, further including presenting, by a second input GUI, the optimized care plan for each new patient to a care manager.

Various embodiments are described, wherein the second input GUI is configured to receive patient specific constraints for new patients from a care manager.

Various embodiments are described, wherein the optimization module uses mixed-integer optimization to determine the optimized care plan for each patient cohort.

Various embodiments are described, wherein the new care plan optimization module uses mixed-integer optimization to determine the optimized care plan for each patient cohort.

### BRIEF DESCRIPTION OF THE DRAWINGS

In order to better understand various exemplary embodiments, reference is made to the accompanying drawings, wherein:

FIG. 1 illustrates an embodiment of a care plan tool;

FIG. 2 illustrates a pop-up box in a GUI that may be used to select the cohort; and

FIG. 3 illustrates an embodiment of the second GUI.

To facilitate understanding, identical reference numerals have been used to designate elements having substantially the same or similar structure and/or substantially the same or similar function.

### DETAILED DESCRIPTION

The description and drawings illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be for pedagogical purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or (i.e., and/or), unless otherwise indicated (e.g., “or else” or “or in the alternative”). Also, the various embodiments described herein are not necessarily mutually exclusive, as some embodiments can be combined with one or more other embodiments to form new embodiments.

Descriptive, diagnostic, and predictive analytics have been used significantly to understand the healthcare system. For example, the risk of re-admission, ED visits, mortality, and annual healthcare expenditure has been widely studied. However, to better design and optimize the healthcare system, prescriptive studies may be performed for example to determine how to best use limited resources to treat patients that will provide the most benefit for the expenditure of the resources.

For example, one of the main challenges for a care manager director is to decide how to allocate their resources to different patients e.g., how many times to call patient A within the next month, how many times to visit patient B's home within the next month, how much money to invest on the care plan of patient C, etc. Suppose a care manager director in an emergency department has a budget to spend $100K (in terms of nurses' time, commute cost, phone calling cost) on the population of ED patients who have visited the emergency department in the past month with a goal to reduce the risk that the patients return to the ED within one month of their discharges. How should the care manager director allocate the resources?

Embodiments of a care plan tool to allocate the resources to different patients in the portfolio of the care manager director will be described herein. The care plan tool described will be for an ED, but the tool may be applied to other medical departments and applications as well. Also, each care manager under the supervision of the care manager director has the flexibility to either accept the care plans for their patients or try to adjust the optimization procedure based on their intuition/insights, considering that the care manager has a fixed amount of resources available based on the first optimization process by the care manager director. For example, care manager A manages 100 patients and based on a first optimization procedure carried out by the care plan tool under the direction of the care manager director, and care manager A may spend $20 k within the next month (in terms of 2 nurse full time employees (FTEs), $2 k for commuting costs, and $3 k for phone calls) on their patient portfolio. The care manager can either accept their patient care plans (output of the optimization process of the care plan tool as directed by the care manager director) or change the rules/constraints to better invest the allocated $20 k. The care plan tool provides the ability to optimize the care management resource allocations in a health care facility such as an emergency department. Using this care plan tool, care plans may be suggested and tailored for each individual ED patient, considering the logistic/staff/funding constraints, so that their risk of ED return visits is reduced.

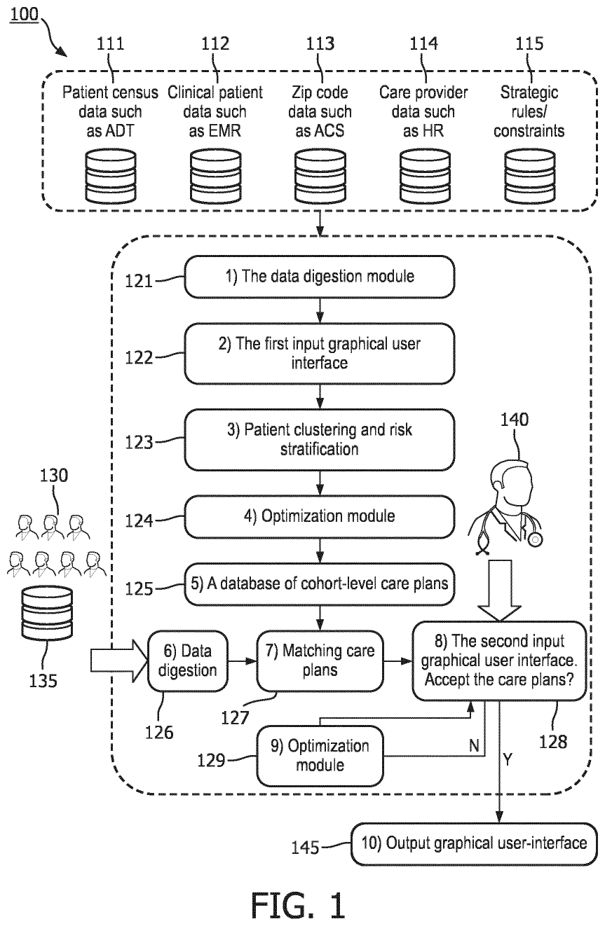

FIG. 1 illustrates an embodiment of a care plan tool. The care plan tool 100 may include a data digestion module 121, a first input graphical user interface (GUI) 122, a patient clustering and risk stratification module 123, an optimization module 124, a database of cohort-level care plans 125, patient data digestion module 126, matching care plans module 127, second GUI 128, new care plan optimization module 129, and an output GUI 145. Each of these will be described further below.

The data digestion module 121 reads in the patient census data 111 (such as admit/discharge/transfer ADT data), clinical patient data 112 (such as electronic medical records (EMR)), zip code data 113 (such as American community survey (ACS) data), care provider data 114 (such as human resources (HR) data), strategic rules/constraints data 115 (such as funding constraint, full-time employee (FTE) constraints) for the emergency department. Patient census data 111 may be used to understand what and how many patients visited the emergency or other departments over the last month. The clinical patient data 112 may be used to obtain the clinical view of patients as the clinical patient data 112 includes a wide variety of data collected for the patient. The zip code 113 data may be used to obtain social determinants of health (SDoH) indices of patients as various research has shown that various SDoH factors correspond to geographic location. The care provider data 114 are used to understand how many nurses and or other medical personnel are available now to provide care to patients. The strategic rules/constraints data 115 may be used to understand how much money the care manager director may spend, how many nurses they may assign for visiting patients' homes, etc. For example, the ED department can put constraints on: (1) how much money the care manager director may spend e.g., the whole budget is $100 k; (2) how many nurses may be assigned for visiting patients' homes e.g., half of the nurses are allowed to visit patients at home; (3) the number of home visits per month that a nurse can have e.g., the time of each nurse spent on home visits must not exceed 20% of their work time; (4) when a nurse can do home visits e.g., there must not be any home visits in December; (5) work schedule of nurses e.g., the nurses must not work two consecutive weekends.

The first input GUI module 122 may be used by the care manager director to enter as many additional rules/constraints/preferences in the optimization module as they wish. For example, the care manager director may reduce funding for some specific chronic condition patients and increase it for others, e.g., increasing the budget for CHF patients by 20%. They may enter the maximum number of home visits that their emergency department staff can do within the next month, e.g., due to weather condition, preferring follow-up phone calls over home visits (number of phone calls must be at least twice the number of home visits). They can specify a certain number of phone visits before a home visit is carried out. This first input GUI module 122 may allow for the input of rules with certain templates to be selected by the user where the data values and/or operators used in the template are selectable. Further, there may be an option for the care manager director to input a Boolean expression of using various data variables to allow for precise definition of care plan rules.

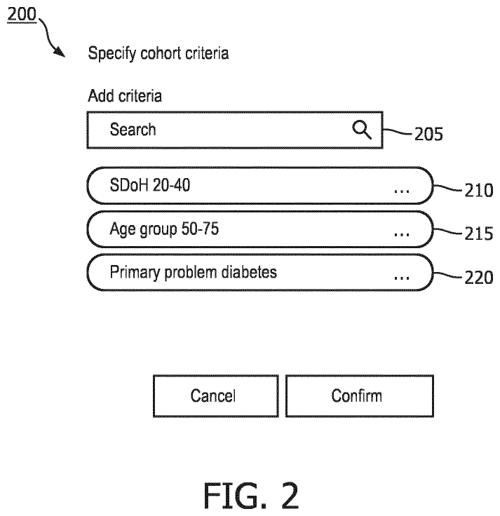

A patient clustering and risk stratification module 123 first clusters patients using the top predictors of their ED return visits to identify patient cohorts. Here, the top predictors may be obtained by training a machine learning model such as random forest to predict ED return visits and ranking the predictors in terms of their importance level. For example, the cohort criteria could be based on SDoH, Age group, and primary diagnosis. FIG. 2 illustrates a pop-up box 200 in a GUI that may be used to select the cohort. The pop-up box 200 may include a search box 205 that allows the care manager director to search for desired cohort criteria to be used to select patients for the patient cohort and to select those cohort criteria to include in the pop-up box 200. The pop-up box 200 further shows drop down menus for SDoH 210, age group 215, and primary problem 220. The care manager director clicks on each drop-down menu to select a specific value for the cohort criteria associated with the drop-down menu.

Once the cohort selection criteria have been selected, the patient clustering and risk stratification module 123 then uses the cohort data to produce a desired prediction model. For example, the model may predict the risk of ED return visits for each cohort. Here, the predictors are care manager-patient interactions such as home visits, phone calls, text messages, etc. Then, the model predicts the risk of ED return visits for each cohort using machine learning models such as logistic regression. This prediction model based upon the cohort data will be used in the optimization module 124.

The optimization module 124 derives optimal care plans for each cluster of patients as produced by the patient clustering and risk stratification module 123 by solving a mixed-integer programming problem to reduce the risk of ED return visits, considering the defined constraints from the data digestion module 121 and the first input GUI 122. To do so, the return to ED probability for a given plan is calculated using machine learning model produced by the patient clustering and risk stratification module 123, where the machine learning model is trained for each cohort separately. Hence, the logistic regression model for each cohort could be as follows:

Risk of ED return visits (within 30 days)=1/(1+exp(−β₀^{c}−β₁^{c}×(#of home visits)−β₂^{c}×(#of phone calls)−β₃^{c}×(#of text messages)))

where βᵢ^{c} is the i^{th} coefficient of the logistic regression model corresponding to the c^{th} cohort.

Suppose the care manager director has $100 k to spend for 1000 ED patients who visited their ED over the last month. Further, assume that the care manager director cannot spend more than 10 FTEs of nursing for home visits, 5 FTEs for phone calls and text messages, etc. What should the care plans for each cohort be considering the constraints?

For example, consider the following care items: home visits, phone calls and text messages. Let x₁, x₂ and x₃ denote the number of times that home visits, phone calls, and text messages are used respectively. The probability of returning to ED based upon a machine learning model trained using the cohort data for each cohort could be as follows.

For example, for the first cohort of Diabetes patients with 50<age≤75 and 20<SDoH index≤40, the risk of ED return visits within 30 days is as follows:

$$Pr ( return to ED within 30 days \vert cohort = \{ Diabetes , 50 < age \leq 75 and 20 < SDoH index \leq 40 \} ) = \frac{1}{1 + {e}^{- ( 0.6 - 0.1 * {x}_{1}^{1} - 0.05 * {x}_{2}^{1} - 0.07 * {x}_{3}^{1} )}}$$

where xᵢ^{c} is the i^{th} care item of the logistic regression model corresponding to the c^{th} cohort.

While for the second cohort of CHF patients with 50<age≤75 and 20<SDoH index≤40, the risk of ED return visits within 30 days is as follows:

$$Pr ( return to ED within 30 days \vert cohort = \{ CHF , 50 < age \leq 75 and 20 < SDoH index \leq 40 \} ) = \frac{1}{1 + {e}^{- ( 0.4 - 0.08 * {x}_{1}^{2} - 0.03 * {x}_{2}^{2} - 0.05 * {x}_{3}^{2} )}}$$

The objective function in the Mixed-integer programming problem is the summation of all of these risk of ED return visits for each cohort. Suppose there are N^{c} patients in the c^{th} cohort.

Let Fᵢ, be the cost of intervention i, the goal is to find the values of xᵢ^{c} hat minimize the probability of return for all patients:

$$Min {\sum }_{c} {N}^{c} \times 1 \text{/} ( 1 + exp ( - {β}_{0}^{c} - {β}_{1}^{c} \times {x}_{1}^{c} - {β}_{2}^{c} \times {x}_{2}^{c} - {β}_{3}^{c} \times {x}_{3}^{c} ) )$$

Subject to:

xᵢ^{c}=0,1,2,3, . . . for i=1,2,3
Σᵢ₌₁³Fᵢ×Σ_{c}N^{c}×xᵢ^{c}<100 k, which means the whole budget is $100 k, and
Σ_{c}N^{c}×x₁^{c}≤100, which means there must not be more than 100 home visits etc.

This mixed-integer programming problem may be solved by many different optimization solvers such as CPLEX.

Once the various care plans have been defined by the optimization module 124, the care plans are stored in a database of cohort-level care plans 125. Accordingly, each patient cohort will have a care plan associated with it that is stored in the database of cohort-level care plans 125.

The patient data digestion module 126 reads in the clinical and ADT data for the patients 130 associated with a care manager 140, who is supervised by the care manager director. Suppose the care manager director has 1000 ED patients and ten managers who manage this portfolio of patients. For example, a care manager who has 100 patients uses the tool to pull in the clinical and ADT data of the 100 patients 130. The patient direction module 126 receive inputs from the care manager 140 and then queries the patient data base 135 to collect all of the clinical and ADT for the patients 130. Next, the matching care plans module 127 matches each of the patients 130 to the different cohorts defined by the patient clustering and risk stratification module 123. This may be accomplished by using various methods e.g., we can utilize the top predictors mentioned in the pop-up box 200 to match patients in module 135 to the care plans in module 125. Then the care plan associated with the cohort is assigned to the patients 130.

Using the second GUI 128 the care manager may either accept the care plans for their assigned patients or reject them and adjust the constraints for each patient, considering that the care manager has a fixed amount of resources. For example, Care manager A knows from Module 7 that they can spend $20 k within the next month (in terms of 2 nurse FTEs, $2 k for commute cost, $3 k for phone calls) on their patient population. Here, the care manager can change constraints for some patients based on their intuition/insights regarding specific patients under their care, which allows for a more tailored care plan for each individual patient. For example, a care manager may know that a specific patient does not want home visits and would better respond to phone calls and that another patient does not answer the phone but responds well to home visits.

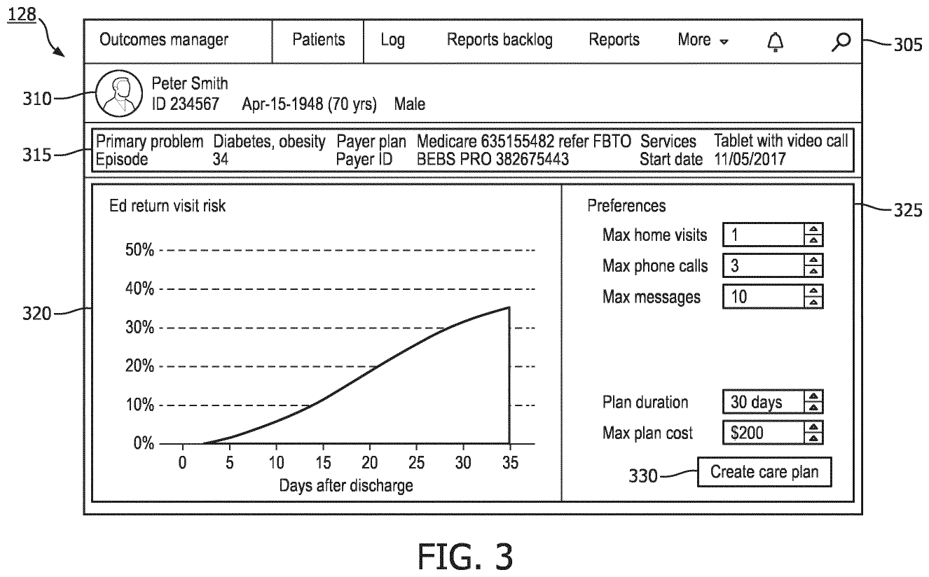

FIG. 3 illustrates an embodiment of the second GUI 128. The GUI 128 may include a header pane 305, a patient identification pane 310, patient medical information pane 315, patient plot pane 320, and a patient preferences pain 325. The header pane 305 may include various tabs that may be selected by the care manager 140 to display various information in the GUI such as Patients, Log, Reports Backlog, Reports, More, etc. The patient identification pane 310 may include various information about the patient, such as for example, a picture, name, ID number date of birth, gender, etc. The patient medical information pane 315 may contain relevant medical information that may be used by the care manager 140 to make care decisions for the patient. Such information may include the Primary problem, Episode number, Payer plan, Payer ID, Services, and Start date. The patient plot pane 320 may include a plot showing for example the risk of a return ED visit versus the number of days after discharge. The care manager 140 may use this plot to determine if further changes to the care plan for the specific patient is warranted. Further, other relevant information may be presented in patient plot pane depending on the issues being addressed by the care plan. The patient preferences pane 325 presents the various care plan items under consideration such as maximum home visits, maximum phone calls and maximum messages, plan duration, and plan cost. Initially, these values are based upon the optimize plan obtained for the patient cohort as described above. At this point the care manager 140 can modify these values for the specific patient based upon the care managers intuition/insights regarding the specific patient.

The new care plan optimization module 129 calculates the new optimal care plans for each patient by solving a mixed-integer programming problem to reduce the risk of ED return visits, considering the new constraints for various patients input by the care manager 140 using the second GUI 128. This optimization may be performed like the optimization described above for the optimization module 124. The only difference is that here the optimization constraints are defined for individual patients, rather than the cohorts of patients. The new optimized care plans are then displayed on the second GUI 128 for review, and if desired the care manager 140 may make further changes to the care plan constraints.

Once, the care manager accepts the care plans assigned to their patient portfolio, the output GUI 145 displays the care plans assigned to each patient the care manager. At this point additional actions may be initiated by the care manager 140 to have the care plan implemented.

The embodiments described herein solve the technological problem of selecting care plans for a group of patients when there are constrained resources available to implement the care plans. These embodiments allow for a care giver to determine how to best utilize funds constrained resources to provide the most benefit to a group of patients.

The embodiments described herein may be implemented as software running on a processor with an associated memory and storage. The processor may be any hardware device capable of executing instructions stored in memory or storage or otherwise processing data. As such, the processor may include a microprocessor, field programmable gate array (FPGA), application-specific integrated circuit (ASIC), graphics processing units (GPU), specialized neural network processors, cloud computing systems, or other similar devices.

The memory may include various memories such as, for example L1, L2, or L3 cache or system memory. As such, the memory may include static random-access memory (SRAM), dynamic RAM (DRAM), flash memory, read only memory (ROM), or other similar memory devices.

The storage may include one or more machine-readable storage media such as read-only memory (ROM), random-access memory (RAM), magnetic disk storage media, optical storage media, flash-memory devices, or similar storage media. In various embodiments, the storage may store instructions for execution by the processor or data upon with the processor may operate. This software may implement the various embodiments described above including implementing the care plan tool that may include a data digestion module, a first input GUI, a patient clustering and risk stratification module, an optimization module, a database of cohort-level care plans, patient data digestion module, matching care plans module, a second GUI, a new care plan optimization module, and an output GUI.

Further such embodiments may be implemented on multiprocessor computer systems, distributed computer systems, and cloud computing systems. For example, the embodiments may be implemented as software on a server, a specific computer, on a cloud computing, or other computing platform.

Any combination of specific software running on a processor to implement the embodiments of the invention, constitute a specific dedicated machine.

As used herein, the term “non-transitory machine-readable storage medium” will be understood to exclude a transitory propagation signal but to include all forms of volatile and non-volatile memory.

Although the various exemplary embodiments have been described in detail with particular reference to certain exemplary aspects thereof, it should be understood that the invention is capable of other embodiments and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

What is claimed is:

1. A care plan tool for defining care plans for patients with constrained care plan resources, comprising:
a patient clustering and risk stratification module configured to cluster a group of patients into patient cohorts based upon cohort criteria and configured to produce a machine learning model to predict the risk of a medical condition for each patient cohort based upon medical data for the patients in the cohort;
an optimization module configured to determine an optimized care plan for each patient cohort based upon the machine learning models for each patient cohort and the constrained care plan resources;
a matching care plan module configured to receive patient data for new patients and configured to match the new patients to a patient cohort and associated care plan; and
a new care plan optimization module configured to receive patient specific constraints for new patients from a care manager and configured to determine a new optimized care plan for each new patient based upon the machine learning models for each patient cohort, the constrained care plan resources, patient data for new patients, and patient specific constraints.

2. The care plan tool of claim 1, further comprising a first input GUI configured to receive from a care manager one of rules, constraints, and preferences to define the constrained care plan resources.

3. The care plan tool of claim 1, further comprising a data digestion module configured to obtain patient data from a patient database.

4. The care plan tool of claim 3, wherein the patient database includes one of electronic medical records, zip code data, care provider data, and constraints data.

5. The care plan tool of claim 1, further comprising a database of optimized care plan for each patient cohort.

6. The care plan tool of claim 1, further comprising a patient data digestion model configured to extract patient data for new patients from a patient data base.

7. The care plan tool of claim 1, further comprising a second input GUI configured to present the optimized care plan for each new patient to a care manager.

8. The care plan tool of claim 7, wherein the second input GUI is configured to receive patient specific constraints for new patients from a care manager.

9. The care plan tool of claim 1, wherein the optimization module uses mixed-integer optimization to determine the optimized care plan for each patient cohort.

10. The care plan tool of claim 1, wherein the new care plan optimization module uses mixed-integer optimization to determine the optimized care plan for each patient cohort.

11. A method for defining care plans for patients with constrained care plan resources, comprising:
clustering, by a patient clustering and risk stratification module, a group of patients into patient cohorts based upon cohort criteria;
producing, by the patient clustering and risk stratification module a machine learning model to predict the risk of a medical condition for each patient cohort based upon medical data for the patients in the cohort;
determining, by an optimization module, an optimized care plan for each patient cohort based upon the machine learning models for each patient cohort and the constrained care plan resources;
receiving, by a matching care plan module, patient data for new patients;
matching, by a matching care plan module, the new patients to a patient cohort and associated care plan;
receiving, by a new care plan optimization module, patient specific constraints for new patients from a care manager; and
determining, by a new care plan optimization module, a new optimized care plan for each new patient based upon the machine learning models for each patient cohort, the constrained care plan resources, patient data for new patients, and patient specific constraints.

12. The method of claim 11, further comprising receiving, by The method of claim 11, from a care manager one of rules, constraints, and preferences to define the constrained care plan resources.

13. The method of claim 11, further comprising obtaining, by a data digestion module, patient data from a patient database.

14. The method of claim 13, wherein the patient database includes one of electronic medical records, zip code data, care provider data, and constraints data.

15. The method of claim 11, the optimized care plan for each patient cohort is stored in a database of optimized care plans.

16. The method of claim 11, further comprising extracting, by a patient data digestion model, patient data for new patients from a patient data base.

17. The method of claim 11, further comprising presenting, by a second input GUI, the optimized care plan for each new patient to a care manager.

18. The method of claim 17, wherein the second input GUI is configured to receive patient specific constraints for new patients from a care manager.

19. The method of claim 11, wherein the optimization module uses mixed-integer optimization to determine the optimized care plan for each patient cohort.

20. The method of claim 11, wherein the new care plan optimization module uses mixed-integer optimization to determine the optimized care plan for each patient cohort.
